(19) United States
(12) Patent Application Publication
(10) Pub. No.: US 2008/0181948 A1
(43) Pub. Date: Jul. 31, 2008

(54) SOLID PHARMACEUTICAL DOSAGE FORMULATIONS

(75) Inventors: Gunther Berndl, Herxheim (DE); Joerg Rosenberg, Ellerstadt (DE); Bernd Liepold, Dossenheim (DE); Katja Fastnacht, Mannheim (DE); Tina Jung, Mannheim (DE); Wolfgang Roth, Wattenheim (DE); Joerg Breitenbach, Mannheim (DE); John Morris, Grayslake, IL (US); Cheri E. Klein, Northbrook, IL (US); Yan Cai, Lake Bluff, IL (US); Laman Alani, Foster City, CA (US); Soumojeet Ghosh, Landsdale, PA (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 11/939,640

(22) Filed: Nov. 14, 2007

Related U.S. Application Data
(60) Provisional application No. 60/859,271, filed on Nov. 15, 2006.

(51) Int. Cl. A61K 9/28; A61K 31/427; A61P 31/18; A61K 9/107

(52) U.S. Cl. 424/474; 514/365; 424/484

(57) ABSTRACT

The present invention features solid pharmaceutical dosage formulations comprising ritonavir. As a non-limiting example, a dosage form of the present invention comprises a solid dispersion or solid solution of ritonavir in a matrix, where the matrix comprises at least one water-soluble polymer, such as copovidone, and at least one surfactant, such as polyoxyl 40 hydrogenated castor oil or macrogolglycerol hydroxystearate. Preferably, the solid dispersion or solution does not include, or includes only an insignificant amount of, PEG.

This application claims the benefit of U.S. Provisional Application Ser. No. 60/859,271, filed Nov. 15, 2006.

### FIELD OF THE INVENTION

The present invention relates to solid pharmaceutical dosage formulations comprising ritonavir or a combination of ritonavir and another therapeutic agent.

### BACKGROUND

Ritonavir, (2S,3S,5S)-5-(N-(N-((N-methyl-N-((2-isopropyl-4-thiazolyl)methyl)amino)carbonyl)-L-valinyl)amino)-2-(N-((5-thiazolyl)methoxycarbonyl)amino)-1,6-diphenyl-3-hydroxyhexane, is an HIV protease inhibitor. See U.S. Pat. No. 5,541,206, which is incorporated herein by reference in its entirety. Ritonavir is poorly water-soluble and very difficult to formulate. A widely used ritonavir dosage form is gelatin capsule containing a fill solution in which ritonavir is dissolved. Ritonavir gelatin capsules require refrigerated storage conditions to prevent degradation of the active ingredient. For subjects residing in economically challenged or developing countries, such storage conditions represent a particularly challenging dilemma.

### SUMMARY OF THE INVENTION

The present invention features solid pharmaceutical dosage forms comprising a solid dispersion or solid solution of ritonavir in a matrix. Ritonavir accounts for at least 10 wt % of the solid dispersion/solution. The matrix includes at least one pharmaceutically acceptable water-soluble polymer and at least one pharmaceutically acceptable surfactant having an HLB value of from 12 to 18. Where the solid dispersion/solution comprises two or more surfactants, at least 50 wt % of all surfactants, based on the total weight of all surfactants in the solid dispersion/solution, have an HLB value(s) of from 12 to 18. Preferably, the solid dispersion/solution does not contain, or contains only an insignificant amount of, PEG 6000. More preferably, the solid dispersion/solution does not contain any, or contains only an insignificant amount of, polyethylene glycol (PEG).

It was surprisingly found that, when the same amount of ritonavir was formulated, representative dosage forms of the present invention and the gelatin capsule formulation were bioequivalent or had similar pharmacokinetic profiles. This would allow the development of ritonavir dosage forms that are stable at room temperature and therefore do not require refrigeration for storage.

In one aspect, the present invention features solid dosage forms comprising a solid dispersion/solution of ritonavir in a matrix, where the matrix comprises one or more pharmaceutically acceptable water-soluble polymers in an amount of at least 50 wt %, based on the weight of the solid dispersion/solution. Preferably, the water-soluble polymer (or polymers) is present in an amount of from 50 to 90 wt %, from 60 to 80 wt %, or from 65 to 75 wt %, based on the weight of the solid dispersion/solution. Water-soluble polymers suitable for the present invention include those with T_{g}s of at least 50° C., such as at least 60° C. or from 80 to 180° C. A non-limiting example of suitable water-soluble polymers is copovidone.

The matrix also comprises one or more pharmaceutically acceptable surfactants each of which has an HLB value of from 12 to 18. The surfactant (or surfactants) is present in an amount of at least 1 wt % (e.g., at least 2, 3, 4 or 5 wt %), based on the weight of the solid dispersion/solution. Preferably, the surfactant (or surfactants) has an HLB value of from 13 to 17 or from 14 to 16, and is present in an amount of from 5 to 25 wt %, from 5 to 15 wt %, from 5 to 10 wt %, or from 10 to 15 wt %, based on the weight of the solid dispersion/solution. A non-limiting example of suitable surfactants is polyoxyl 40 hydrogenated castor oil or macrogolglycerol hydroxystearate. Where the matrix comprises two or more surfactants, at least 50 wt % of all surfactants in the matrix have an HLB value(s) of from 12 to 18. In many cases, more than 60, 70, 80, 90, 95, 99 or more wt % of all surfactants in the matrix have an HLB value(s) of from 12 to 18.

In one embodiment, a dosage form of the present invention comprises a solid dispersion/solution of ritonavir in a matrix, wherein ritonavir is present in an amount of from 10 to 30 wt %, and the matrix comprises one or more pharmaceutically acceptable water soluble polymers in an amount of from 50 to 85 wt % and one or more pharmaceutically acceptable surfactants in an amount of from 5 to 20 wt %, each said surfactant having an HLB value of from 12 to 18 and all wt % being based on the weight of the solid dispersion/solution.

In another embodiment, a dosage form of the present invention comprises a solid dispersion/solution of ritonavir in a matrix, wherein ritonavir is present in an amount of from 15 to 25 wt %, and the matrix comprises one or more pharmaceutically acceptable water soluble polymers in an amount of from 65 to 75 wt % and one or more pharmaceutically acceptable surfactants in an amount of from 5 to 15 wt %, each said surfactant having an HLB value of from 12 to 18 and all wt % being based on the weight of the solid dispersion/solution.

In still another embodiment, a dosage form of the present invention comprises a solid dispersion/solution of ritonavir in a matrix, wherein ritonavir is present in an amount of from 10 to 25 wt %, and the matrix comprises copovidone in an amount of from 60 to 80 wt % and polyoxyl 40 hydrogenated castor oil or macrogolglycerol hydroxystearate in an amount of from 5 to 15 wt %, all wt % being based on the weight of the solid dispersion/solution.

In yet another embodiment, a dosage form of the present invention comprises a solid dispersion/solution of ritonavir in a matrix, wherein ritonavir is present in an amount of from 15 to 20 wt %, and the matrix comprises copovidone in an amount of from 65 to 75 wt % and polyoxyl 40 hydrogenated castor oil or macrogolglycerol hydroxystearate in an amount of 10 wt %, all wt % being based on the weight of the solid dispersion/solution.

Preferably, a dosage form of the present invention does not contain any significant amounts of ritonavir in crystalline or microcrystalline state, as evidenced by thermal analysis (DSC) or X-ray diffraction analysis (WAXS). For instance, ritonavir in the dosage form can be dissolved or molecularly dispersed in the matrix.

A solid dispersion/solution of the present invention can also contain one or more glidants, such as colloidal silica. In one example, the solid dispersion/solution comprises at least one glidant, such as colloidal silica, in an amount of from 0.5 to 3 wt %, based on the weight of the solid dispersion/solution. In another example, the solid dispersion/solution comprises at least one glidant, such as colloidal silica, in an amount of 1 wt %, based on the weight of the solid dispersion/solution.

In many embodiments, the solid dispersions/solutions of the present invention do not contain any surfactants that have HLB values of from 4 to 10. In many other embodiments, the solid dispersions/solutions of the present invention contain only an insignificant amount of surfactant(s) that has HLB value(s) of from 4 to 10. As used herein, a component in a dosage form is in an “insignificant” amount if the dosage form is bioequivalent to another dosage form which has the same composition as the former dosage form but without the component at issue (e.g., when tested in humans, the 90% confidence intervals of the relative average Cₘₐₓ, AUCₜ and AUC_{∞} of the former dosage form as compared to the latter dosage are within the range of from 80% to 125%). Non-limiting examples of surfactants having HLB values of from 4 to 10 are described in U.S. Patent Application Publication No. 2005/048112, which is incorporated herein by reference in its entirety. In one example, the solid dispersion/solution in a dosage form of the present invention does not contain sorbitan monolaurate or Span® 20. In another example, the solid dispersion/solution contains only an insignificant amount of sorbitan monolaurate or Span® 20.

In still many embodiments, the total amount of surfactant(s) with an HLB value of from 4 to 10 in a solid dispersion/solution is less than 4, 3, 2, 1, 0.5, 0.1, or 0.01 wt % of the solid dispersion/solution. For instance, a solid dispersion/solution may contain sorbitan monolaurate or Span® 20 in an amount of less than 4, 3, 2, 1, 0.5, 0.1, or 0.01 wt %, based on the total weight of the solid dispersion/solution.

In still yet many embodiments, a solid dispersion/solution of the present invention does not contain, or contains only an insignificant amount of, polyethylene glycol (PEG). Non-limiting examples of PEGs include those with molecular weights ranging from 400 to 8000, such as PEG 600, PEG 1000, PEG 1500, PEG 3000, PEG 4000, PEG 6000 or PEG 7000.

In one embodiment, a solid dispersion/solution of the present invention does not comprise, or comprises only an insignificant amount of, PEG 6000.

In another embodiment, all PEGs in a solid dispersion/solution of the present invention constitute less than 5, 4, 3, 2, 1, 0.5, 0.1, or 0.01 wt % of the solid dispersion/solution.

In yet another embodiment, a solid dispersion/solution of present invention contains less than 5, 4, 3, 2, 1, 0.5, 0.1, or 0.01 wt % of PEG 6000.

Ritonavir in a solid dispersion/solution of the present invention can be, without limitation, non-crystalline ritonavir (e.g., molecularly dispersed ritonavir), crystalline ritonavir, or a mixture thereof. Exemplary ritonavir crystalline forms are depicted in U.S. Pat. No. 6,894,171 and U.S. Patent Application Publication No. 2004/0024031, both of which are incorporated herein by reference in their entireties. In one embodiment, at least 50% of all ritonavir in a solid dispersion/solution of the present invention is non-crystalline ritonavir. In another embodiment, at least 60%, 70%, 80% or 90% of all ritonavir in a solid dispersion/solution of the present invention is non-crystalline ritonavir. In still another embodiment, at least 95%, 96%, 97%, 98%, 99% or 100% of all ritonavir in a solid dispersion/solution of the present invention is non-crystalline ritonavir.

Preferred solid dispersions/solutions of the present invention comprise ritonavir dissolved or molecularly dispersed in a matrix. In many cases, at least 50%, 60%, 70%, 80%, 90%, 95%, 96%, 97%, 98%, 99% or 100% of all ritonavir in a solid dispersion/solution is molecularly dispersed or dissolved in a matrix.

In one embodiment, the solid dispersion/solution of the present invention is mixed with one or more additional excipients, such as calcium hydrogen phosphate or colloidal silica. The mixture can be further compressed into a tablet and coated by a film coating.

A dosage form of the present invention can include, by way of illustration and not limitation, at least 10 mg ritonavir, such as at least 15, 20, 25, or 30 mg ritonavir. In one embodiment, a dosage form of the present invention includes from 10 mg to 1,000 mg, from 50 to 800 mg, from 50 to 400 mg, from 100 to 200 mg, or from 75 to 150 mg ritonavir. In another embodiment, a dosage form of the present invention includes 10, 15, 20, 25, 30, 35, 40, 45, 50, 55, 60, 65, 70, 75, 80, 85, 90, 95, 100, 105, 110, 115, 120, 125, 130, 135, 140, 145, or 150 mg ritonavir.

Preferably, a solid dosage form of the present invention is bioequivalent (when tested in humans) to a reference ritonavir solution which contains the same absolute amount of ritonavir as the solid dosage form. The reference ritonavir solution consists of 12 wt % ethanol, 0.025 wt % butylated hydroxytoluene, 70.975 wt % oleic acid, 10 wt % ritonavir, 1 wt % water, and 6 wt % polyoxyl 35 castor oil. In some cases, the 90% confidence interval of the relative average Cₘₐₓ, AUCₜ or AUC_{∞} of a solid dosage form of the present invention as compared to the reference ritonavir solution is within the range of from 80% to 125%. In some other cases, the 90% confidence intervals of the relative average AUCₜ and AUC_{∞} (or Cₘₐₓ and AUCₜ; or Cₘₐₓ and AUC_{∞}) of a solid dosage form of the present invention as compared to the reference ritonavir solution are within the range of from 80% to 125%. More preferably, the 90% confidence intervals of the relative average Cₘₐₓ, AUCₜ and AUC_{∞} of a solid dosage form of the present invention as compared to the reference ritonavir solution are within the range of from 80% to 125%. AUCₜ can be, for example, AUC from time 0 to 36 hours (i.e., AUC_{36 hours}).

The present invention also features processes of making the dosage forms of the present invention. These processes typically comprise converting a mixture of ritonavir and additional ingredients into a solid dispersion/solution of the present invention, where the additional ingredients include at least one water-soluble polymer and at least one surfactant. The conversion may include solidifying a melt comprising said ritonavir and said additional ingredients. These processes may further comprise grinding the solid dispersion/solution, mixing the ground solid dispersion/solution with one or more additional excipients, and/or compressing the mixture into a tablet. The ground solid dispersion/solution can also be compressed into a tablet without mixing with any additional excipient. The tablets thus prepared can be coated with a film coating.

Any solid dispersions/solution or dosage form of the present invention can be prepared by the processes described above. In one example, a solid dispersion/solution thus prepared comprises from 10 to 30 wt % of ritonavir, from 50 to 85 wt % of a water soluble polymer, and from 5 to 20 wt % of a surfactant which has an HLB value of from 12 to 18. In another example, a solid dispersion/solution thus prepared comprises from 15 to 25 wt % of ritonavir, from 65 to 75 wt % of a water soluble polymer, and from 5 to 15 wt % of a surfactant which has an HLB value of from 12 to 18. In still another example, a solid dispersion/solution thus prepared comprises from 10 to 25 wt % of ritonavir, from 60 to 80 wt % of copovidone, and from 5 to 15 wt % of polyoxyl 40 hydrogenated castor oil or macrogolglycerol hydroxystearate. In still yet another example, a solid dispersion/solution thus prepared comprises from 15 to 20 wt % of ritonavir, from 65 to 75 wt % of copovidone, and 10 wt % of polyoxyl 40 hydrogenated castor oil or macrogolglycerol hydroxystearate.

The initial ritonavir used for the preparation of a solid dispersion/solution of the present invention can be amorphous ritonavir, crystalline ritonavir, or a mixture thereof. Non-limiting examples of suitable ritonavir crystalline forms include Form I crystalline ritonavir and Form II crystalline ritonavir, both of which are described in U.S. Pat. No. 6,894,171. Other suitable ritonavir crystalline forms include those described in U.S. Patent Application Publication No. 2004/0024031. Mixtures of ritonavir crystalline forms can also be used. Ritonavir Form II crystals are the preferred starting material for the preparation of solid dispersions/solutions.

The present invention further features methods of treating HIV infection. These methods comprise administering to a human in need of such treatment a dosage form of the present invention. Non-limiting examples of suitable routes and methods of administration are described in U.S. Pat. No. 5,541,206, which is incorporated herein by reference in its entirety. Oral administration is the preferred route of administration.

In another aspect, the present invention features methods for improving the pharmacokinetics, or increasing the plasma level, of a drug which is metabolized by cytochrome P450 monooxygenase (e.g., cytochrome P450 monooxygenase 3A4). These methods generally comprise administering to a human in need of such treatment a combination of the drug and a dosage form of the present invention, thereby improving the pharmacokinetics or increasing the plasma level of the drug in the human being treated. Drugs whose pharmacokinetics or plasma levels may be improved by co-administering ritonavir include, but are not limited to, immunomodulators (e.g., cyclosporine or FK-506), anti-cancer or chemotherapeutic agents (e.g., taxol or taxotere), antibiotics (e.g., clarithromycin, erythromycin, or telithromycin), antivirals (e.g., indinavir, lopinavir, nelfinavir, saquinavir, atazanavir, amprenavir, fosamprenavir, tipranavir, or darunavir), antihistamines (e.g., astemizole, chlorpheniramine, or terfenidine), calcium channel blockers (e.g., amlodipine, diltiazem, felodipine, lercanidipine, nifedipine, nisoldipine, nitrendipine, or verapamil), beta blockers (e.g., carvedilol, S-metoprolol, propafenone, or timolol), antidepressants (e.g., amitriptyline, clomipramine, desipramine, imipramine, or paroxetine),

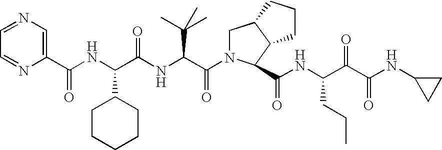

(hereinafter compound VX-950, Vertex Pharmaceuticals Inc.) or a salt, solvate or prodrug thereof,

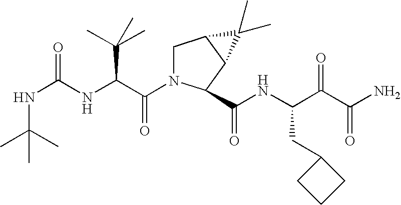

(hereinafter compound SCH503034, Schering-Plough Co.) or a salt, solvate or prodrug thereof,
and

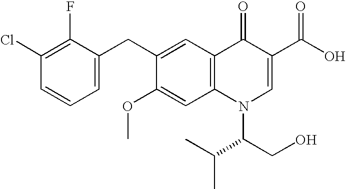

(hereinafter compound GS9137, Gilead Sciences, Inc., Foster City, Calif.) or a salt, solvate or prodrug thereof, and other HIV or HCV protease inhibitors. These drugs can be metabolized by cytochrome P450 monooxygenase (e.g., cytochrome P450 monooxygenase 3A4). Non-limiting examples of other drugs whose pharmacokinetics or plasma levels can be improved by co-administering ritonavir are described in U.S. Pat. Nos. 6,037,157 and 6,703,403, both of which are incorporated herein by reference in their entireties. These drugs can be prepared in a different formulation and administered, either simultaneously or sequentially, with a dosage form of the present invention that comprises ritonavir. These drugs can also be co-formulated with ritonavir in a solid dispersion/solution of the present invention. In addition, these drugs can be prepared in a separate solid dispersion/solution or in another form, and then mixed with a solid dispersion/solution of ritonavir to create a single dosage form.

The present invention also features methods of inhibiting cytochrome P450 monooxygenase (e.g., cytochrome P450 monooxygenase 3A4). The methods comprise administering to a human in need thereof a dosage form of the present invention, thereby inhibiting cytochrome P450 monooxygenase activities in said human.

Other therapeutic agent(s) can also be included in a dosage form of the present invention. These therapeutic agent(s) and ritonavir can be molecularly dispersed in the same solid dispersion/solution. These therapeutic agent(s) can also be formulated separately, and then combined with a solid dispersion/solution of ritonavir to form a single dosage form.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### BRIEF DESCRIPTION OF THE DRAWINGS

The application incorporates by reference FIG. 1 of U.S. Provisional Application Ser. No. 60/859,271, which is color. The drawings are provided for illustration, not limitation.

FIG. 1 shows ritonavir plasma concentrations versus time after oral administration to humans.

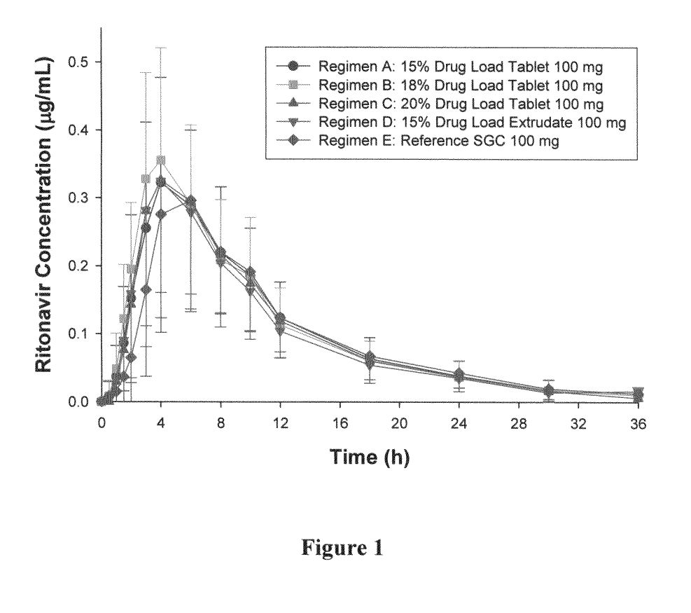

### DETAILED DESCRIPTION

The terms “AUC_{∞}” or “AUC_{inf}” refer to the area under the plasma concentration time curve (AUC) extrapolated to infinity.

The “AUCₜ” refers to AUC from time 0 to the last measured time point. This was approximately 36 hours for most subjects evaluated in the Examples described hereinbelow.

The term “Cₘₐₓ” is defined as the observed maximum plasma concentration of an active ingredient.

The term “CL/F” refers to apparent oral clearance.

T_{1/2} is elimination half-life, i.e., the time taken for plasma concentration to reduce by 50%.

“Pharmaceutically acceptable” as used herein means moieties or compounds that are, within the scope of sound medical judgment, suitable for use in contact with the tissues of humans and lower animals without undue toxicity, irritation, allergic response, and the like, and are commensurate with a reasonable benefit/risk ratio.

The terms “weight percent” or “percent by weight” or “% by weight” or “wt %” denote the weight of an individual component in a composition/mixture/makeup/composite as a percentage of the weight of the composition/mixture/makeup/composite.

The term “solid dispersion” defines a system in a solid state (as opposed to a liquid or gaseous state) comprising at least two components, wherein one component is dispersed throughout the other component or components. For example, the active ingredient or combination of active ingredients is dispersed in a matrix comprised of the pharmaceutically acceptable water-soluble polymer(s) and pharmaceutically acceptable surfactant(s). The term “solid dispersion” encompasses systems having small particles of one phase dispersed in another phase. These particles are typically of less than 400 μm in size, such as less than 100, 10, or 1 μm in size. When said dispersion of the components is such that the system is chemically and physically uniform or homogenous throughout or consists of one phase (as defined in thermodynamics), such a solid dispersion will be called a “solid solution” or a “glassy solution.” A glassy solution is a homogeneous, glassy system in which a solute is dissolved in a glassy solvent.

The present invention features solid dosage forms described hereinabove. Generally, the dosage forms of the present invention will comprise a therapeutically effective amount of ritonavir. The specific therapeutically effective dose level for any particular patient will depend upon a variety of factors including the severity of the disorder; the activity of the specific compound employed; the specific composition employed; the age, body weight, general health, sex and diet of the patient; the time of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved. It will be understood that multiple doses, typically two, can be given in a given day.

Many pharmaceutical dosage forms are acceptable for use in accordance with the present invention, the choice of which is well within the skill of a person of ordinary skill in this art based upon the properties of the dosage forms provided herein. For example, orally administered solid dosage forms include but are not limited to capsules, dragees, granules, pills, powders, and tablets. Excipients commonly used to formulate such dosage forms include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, preservatives, propellants, releasing agents, sterilizing agents, sweeteners, solubilizers, and mixtures thereof. Excipients for orally administered compounds in solid dosage forms include agar, alginic acid, aluminum hydroxide, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, talc, tragacanth, water, safflower oil, sesame oil, sodium carboxymethyl cellulose, sodium lauryl sulfate, sodium phosphate salts, soybean oil, sucrose, tetrahydrofurfuryl alcohol, and mixtures thereof.

A typical dosage form of the invention, including those described hereinabove, comprises a solid solution or solid dispersion of ritonavir in a matrix, wherein the ritonavir is in a therapeutically effective amount, and the matrix comprises at least one pharmaceutically acceptable water-soluble polymer and at least one pharmaceutically acceptable surfactant.

Suitable pharmaceutically acceptable water-soluble polymers include, but are not limited to, water-soluble polymers having a T_{g} of at least 50° C., preferably at least 60° C., more preferably from 80° C. to 180° C. Methods for determining T_{g} values of the organic polymers are described in INTRODUCTION TO PHYSICAL POLYMER SCIENCE (2nd Edition by L. H. Sperling, published by John Wiley & Sons, Inc., 1992). The T_{g} value can be calculated as the weighted sum of the T_{g} values for homopolymers derived from each of the individual monomers, i.e., the polymer T_{g}=ΣWᵢXᵢ where Wᵢ is the weight percent of monomer i in the organic polymer, and Xᵢ is the T_{g} value for the homopolymer derived from monomer i. T_{g} values for the homopolymers may be taken from POLYMER HANDBOOK (2nd Edition by J. Brandrup and E. H. Immergut, Editors, published by John Wiley & Sons, Inc., 1975).

Water-soluble polymers having a T_{g} as defined above allow for the preparation of solid solutions or solid dispersions that are mechanically stable and, within ordinary temperature ranges, sufficiently temperature stable so that the solid solutions or solid dispersions may be used as dosage forms without further processing or be compacted to tablets with only a small amount of tableting aids.

The water-soluble polymer comprised in a preferred dosage form is a polymer that preferably has an apparent viscosity, when dissolved at 20° C. in an aqueous solution at 2% (w/v), of 1 to 5000 mPa·s, and more preferably of 1 to 700 mPa·s, and most preferably of 5 to 100 mPa·s.

Water-soluble polymers suitable for use in a preferred dosage form of the present invention include but are not limited to homopolymers and copolymers of N-vinyl lactams, especially homopolymers and copolymers of N-vinyl pyrrolidone, e.g. polyvinylpyrrolidone (PVP), copolymers of N-vinyl pyrrolidone and vinyl acetate or vinyl propionate; cellulose esters and cellulose ethers, in particular methylcellulose and ethylcellulose, hydroxyalkylcelluloses, in particular hydroxypropylcellulose, hydroxyalkylalkylcelluloses, in particular hydroxypropylmethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate; high molecular polyalkylene oxides such as polyethylene oxide and polypropylene oxide and copolymers of ethylene oxide and propylene oxide; polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates); polyacrylamides, vinyl acetate polymers such as copolymers of vinyl acetate and crotonic acid, partially hydrolyzed polyvinyl acetate (also referred to as partially saponified “polyvinyl alcohol”), polyvinyl alcohol; oligo- and polysaccharides such as carrageenans, galactomannans and xanthan gum, or mixtures of one or more thereof.

Of these, homopolymers or copolymers of N-vinyl pyrrolidone, in particular copolymers of N-vinyl pyrrolidone and vinyl acetate, are preferred. A particularly preferred polymer is a copolymer of 60% by weight of the copolymer, N-vinyl pyrrolidone and 40% by weight of the copolymer, vinyl acetate.

According to a preferred dosage form of the present invention, the pharmaceutical dosage form comprises from 50 to 85% by weight of the total dosage form, preferably from 60 to 80% by weight of the total dosage form, of a water-soluble polymer or any combination of such polymers.

The term “pharmaceutically acceptable surfactant” as used herein refers to a pharmaceutically acceptable non-ionic surfactant. A dosage form of the present invention comprises at least one surfactant having a hydrophilic lipophilic balance (HLB) value of from 12 to 18, preferably from 13 to 17, or more preferably from 14 to 16. The HLB system (Fiedler, H. B., ENCYLOPEDIA OF EXCIPIENTS, 5^{th} ed., Aulendorf: ECV-Editio-Cantor-Verlag (2002)) attributes numeric values to surfactants, with lipophilic substances receiving lower HLB values and hydrophilic substances receiving higher HLB values.

In one embodiment, a dosage form of the invention comprises one or more pharmaceutically acceptable surfactants selected from polyoxyethylene castor oil derivates, e.g. polyoxyethyleneglycerol triricinoleate or polyoxyl 35 castor oil (Cremophor® EL; BASF Corp.) or polyoxyethyleneglycerol oxystearate such as polyethylenglycol 40 hydrogenated castor oil (Cremophor® RH 40, also known as polyoxyl 40 hydrogenated castor oil or macrogolglycerol hydroxystearate) or polyethylenglycol 60 hydrogenated castor oil (Cremophor® RH 60); or a mono fatty acid ester of polyoxyethylene (20) sorbitan, e.g. polyoxyethylene (20) sorbitan monooleate (Tween® 80), polyoxyethylene (20) sorbitan monostearate (Tween® 60), polyoxyethylene (20) sorbitan monopalmitate (Tween® 40), or polyoxyethylene (20) sorbitan monolaurate (Tween® 20). Other surfactants including those with HLB values of greater than 18 or less than 12 may also be used, e.g., block copolymers of ethylene oxide and propylene oxide, also known as polyoxyethylene polyoxypropylene block copolymers or polyoxyethylene polypropyleneglycol, such as Poloxamer® 124, Poloxamer® 188, Poloxamer® 237, Poloxamer® 388, or Poloxamer® 407 (BASF Wyandotte Corp.).

Where two or more surfactants are used, the surfactant(s) having an HLB value of from 12 to 18 preferably accounts for at least 50% by weight, more preferably at least 60% by weight, of the total amount of surfactants used.

A dosage form of the present invention can include additional excipients or additives such as flow regulators, lubricants, bulking agents (fillers) and disintegrants. Such additional excipients may comprise, without limitation, from 0 to 15% by weight of the total dosage form.

A solid dispersion/solution-based dosage form of the present invention can be produced by preparing a solid solution/dispersion of ritonavir, or a solid solution/dispersion of ritonavir and another therapeutic agent(s), in a matrix comprising a water-soluble polymer and a surfactant, and then shaping the solid solution/dispersion into the required tablet form. Alternatively, the solid solution/dispersion can be subdivided to granules, e.g. by grinding or milling, and the granules may subsequently be compacted to tablets.

Various techniques exist for preparing solid solutions or solid dispersions including melt-extrusion, spray-drying and solution-evaporation with melt-extrusion being preferred.

The melt-extrusion process comprises the steps of preparing a homogeneous melt of ritonavir or a combination of ritonavir and another therapeutic agent(s), the water-soluble polymer and the surfactant, and then cooling the melt until it solidifies. “Melting” means a transition into a liquid or rubbery state in which it is possible for one component to get embedded homogeneously in the other. Typically, one component will melt and the active ingredient(s) will dissolve in the melt thus forming a solution. Melting usually involves heating above the softening point of the water-soluble polymer. The preparation of the melt can take place in a variety of ways. The mixing of the components can take place before, during or after the formation of the melt. For example, the components can be mixed first and then melted, or be simultaneously mixed and melted. Usually, the melt is homogenized in order to disperse the active ingredients efficiently. Also, it may be convenient first to melt the water-soluble polymer and then to mix in and homogenize the active ingredients.

Usually, the melt temperature is in the range of 70 to 250° C., preferably from 80 to 180° C., most preferred from 100 to 140° C.

The active ingredients can be employed as such or as a solution or dispersion in a suitable solvent such as alcohols, aliphatic hydrocarbons or esters. Another solvent that can be used is liquid carbon dioxide. The solvent can be removed, e.g. evaporated, upon preparation of the melt.

Various additives may be included in the melt, for example flow regulators (such as colloidal silica), lubricants, fillers, disintegrants, plasticizers, stabilizers such as antioxidants, light stabilizers, radical scavengers, stabilizers against microbial attack.

The melting and/or mixing takes place in an apparatus customary for this purpose. Particularly suitable ones are extruders or kneaders. Suitable extruders include single screw extruders, intermeshing screw extruders or else multiscrew extruders, preferably twin screw extruders, which can be corotating or counterrotating and, optionally, be equipped with kneading disks or other screw elements for mixing or dispersing the melt. It will be appreciated that the working temperatures will also be determined by the kind of extruder or the kind of configuration within the extruder that is used. Part of the energy needed to melt, mix and dissolve the components in the extruder can be provided by heating elements. However, the friction and shearing of the material in the extruder may also provide a substantial amount of energy to the mixture and aid in the formation of a homogeneous melt of the components.

The melt ranges from pasty to viscous. Shaping of the extrudate can be conveniently carried out by a calender with two counter-rotating rollers with mutually matching depressions on their surface. A broad range of tablet forms can be attained by using rollers with different forms of depressions. Alternatively, the extrudate can be cut into pieces, either before (hot-cut) or after solidification (cold-cut).

Optionally, the resulting solid solution or solid dispersion product is milled or ground to granules. The granules may then be compacted. Compacting means a process whereby a powder mass comprising the granules is densified under high pressure in order to obtain a compact with low porosity, e.g. a tablet. Compression of the powder mass is usually done in a tablet press, more specifically in a steel die between two moving punches. Where a solid dosage form of the invention comprises a combination of ritonavir and another active ingredient(s), it is possible to separately prepare solid solution or solid dispersion products of the individual active ingredients and then blend the milled or ground products before compacting.

At least one additive selected from flow regulators, disintegrants, bulking agents (fillers) and lubricants can be used in compacting the granules. These additives can be mixed with ground or milled solid solutions/dispersions before compacting. Disintegrants promote a rapid disintegration of the compact in the stomach and keeps the granules which are liberated separate from one another. Non-limiting examples of suitable disintegrants are crosslinked polymers such as crosslinked polyvinyl pyrrolidone and crosslinked sodium carboxymethylcellulose. Non-limiting examples of suitable bulking agents (also referred to as “fillers”) are lactose, calcium hydrogenphosphate, microcrystalline cellulose (Avicell®), silicates, in particular silicium dioxide, magnesium oxide, talc, potato or corn starch, isomalt, or polyvinyl alcohol. Non-limiting examples of suitable flow regulators include highly dispersed silica (Aerosil®), and animal or vegetable fats or waxes. Non-limiting examples of suitable lubricants include polyethylene glycol (e.g., having a molecular weight of from 1000 to 6000), magnesium and calcium stearates, sodium stearyl fumarate, and the like.

Various other additives may be used, for example dyes such as azo dyes, organic or inorganic pigments such as aluminium oxide or titanium dioxide, or dyes of natural origin; stabilizers such as antioxidants, light stabilizers, radical scavengers, stabilizers against microbial attack.

Dosage forms according to the invention may be provided as dosage forms consisting of several layers, for example laminated or multilayer tablets. They can be in open or closed form. “Closed dosage forms” are those in which one layer is completely surrounded by at least one other layer. Multilayer forms have the advantage that two active ingredients which are incompatible with one another can be processed, or that the release characteristics of the active ingredient(s) can be controlled. For example, it is possible to provide an initial dose by including an active ingredient in one of the outer layers, and a maintenance dose by including the active ingredient in the inner layer(s). Multilayer tablets types may be produced by compressing two or more layers of granules. Alternatively, multilayer dosage forms may be produced by a process known as “coextrusion.” In essence, the process comprises preparation of at least two different melt compositions as explained above, and passing these molten compositions into a joint coextrusion die. The shape of the coextrusion die depends on the required drug form. For example, dies with a plain die gap, called slot dies, and dies with an annular slit are suitable.

In order to facilitate the intake of such a dosage form by a mammal, it is advantageous to give the dosage form an appropriate shape. Large tablets that can be swallowed comfortably are therefore preferably elongated rather than round in shape.

A film coat on the tablet further contributes to the ease with which it can be swallowed. A film coat also improves taste and provides an elegant appearance. If desired, the film-coat may be an enteric coat. The film-coat usually includes a polymeric film-forming material such as hydroxypropyl methylcellulose, hydroxypropylcellulose, and acrylate or methacrylate copolymers. Besides a film-forming polymer, the film-coat may further comprise a plasticizer, e.g. polyethylene glycol, a surfactant, e.g. a Tween® type, and optionally a pigment, e.g. titanium dioxide or iron oxides. The film-coating may also comprise talc as anti-adhesive. The film coat usually accounts for less than 5% by weight of the dosage form.

The benefits provided by the present invention include improving pharmacokinetic (PK) properties. Pharmacokinetic properties are generally understood to mean the manner and extent to which a drug is absorbed. Common PK parameters include AUC (or “area under the curve”), which typically refers to the amount of a drug that is measurable in the blood of a person taking the drug over time. AUC is variously referred to as a patient's exposure to a drug. Cₘₐₓ is another PK term which refers to the maximum blood level of a drug over the course of a given regimen of the drug. Drug regimens for which PK parameters are measured include “clinical studies.” Some clinical studies are performed in a finite population of healthy volunteer patients and are designed to determine the PK parameters of a drug (such as those mentioned above), and not to treat a patient. Each patient is thus called a member of the study population. While such clinical studies are carefully controlled and monitored, PK parameters can vary between clinical studies in large measure because different clinical studies are performed on different populations of patients.

It will be understood that when ritonavir is co-administered with another therapeutic agent(s), they can be administered in separate dosage forms, or in a single dosage form which comprises both ritonavir and the other therapeutic agent(s).

It should be understood that the above-described embodiments and the following examples are given by way of illustration, not limitation. Various changes and modifications within the scope of the present invention will become apparent to those skilled in the art from the present description.

### EXAMPLE 1

The formulations used in this Example were prepared using the melt extrusion processes similar to those described in U.S. Patent Application Publication No. 2005/0048112, which is incorporated herein by reference in its entirety. Generally, copovidone (copolymer of N-vinyl pyrrolidone and vinyl acetate in a ratio of 6:4 by mass) was blended with polyoxyl 40 hydrogenated castor oil (e.g., Cremophor® RH 40), and then mixed with ritonavir and colloidal silica. The powdery mixture was then fed into an extruder at a selected rate (e.g., from 2 to 3 kg/h) and melt temperature (e.g., from 115 to 135° C.). The extrudate can be cut into pieces and allowed to solidify. The extruded pieces were then milled and blended with other excipients such as fillers (e.g., calcium hydrogen phosphate) or glidants (colloidal silica). The powdery blend was compressed to tablets. The tablets were then film-coated. Alternatively, the formulation was extruded in the shape of a tablet, or compressed into a tablet, without the additional processing step of milling.

The extrusion processes for the following formulations used the same excipients but differed in the total drug concentration and relative amounts of excipients. For ritonavir tablet formulation Form E-15, the extrudate was calendered into the final tablet shape, which was subsequently deburred and film-coated. For ritonavir tablet formulation Forms 15, 18 and 20, the extrudate was milled, blended with additional excipients, sieved, blended again and compressed into tablets, which were film-coated. Different amounts of the same tableting excipients are used for these three tablet formulations. The compositions of these ritonavir tablet formulations are presented in Table 1.

TABLE 1
Ritonavir Tablet Formulations

| | Form E-15 | Form 15 | Form 18 | Form 20 |
|---|---|---|---|---|
| Formulation | (mg/tablet) | (mg/tablet) | (mg/tablet) | (mg/tablet) |
| Extrusion | | | | |
| Ritonavir | 100.0 | 100.0 | 100.0 | 100.0 |
| Copovidone (K value 28) | 493.1 | 493.1 | 394.2 | 327.1 |
| Colloidal anhydrous silica | 6.9 | 6.9 | 5.8 | 5.0 |
| Cremophor ® RH 40 | 66.7 | 66.7 | 55.6 | 48.0 |
| Post Extrusion | | | | |
| Calcium hydrogen phosphate, | N/A | 90.2 | 75.1 | 64.9 |
| anhydrous | | | | |
| Colloidal anhydrous silica | N/A | 13.9 | 11.6 | 10.0 |
| Film Coating | | | | |
| Film-coating powder^{a,b} | 18.0 | 18.0 | 18.0 | 14.0 |
| Purified water^{c} | 120.5 | 120.5 | 120.5 | 93.7 |
| Total Tablet Weight | 684.7 | 788.8 | 660.3 | 569.0 |

^{a}film coat weight is approximate, less coating required for Form 20 due to smaller tablet size.
^{b}Opadry ®, Yellow (16B22295), quantitative composition given in Table 2.
^{c}Removed during processing.

TABLE 2
Composition of Film Coating Powder,
Opadry ®, Yellow (16B22295)

| Component | Amount (%, w/w) |
|---|---|
| Hypromellose, 2910 (6 mPa · s) | 58.04 |
| Titanium dioxide | 10.29 |
| Macrogol type 400 | 9.00 |
| Hydroxypropylcellulose | 5.76 |
| Hypromellose, 2910 (15 mPa · s) | 5.76 |
| Talc | 4.10 |
| Colloidal anhydrous silica | 0.15 |
| Macrogol type 3350 | 1.61 |
| Yellow ferric oxide E172 | 5.14 |
| Polysorbate 80 | 0.15 |

The above ritonavir formulations were used in a single dose, non-fasting, four period, partial cross-over, randomised biostudy. Thirty-two (32) healthy adults received 4 of the 5 formulations listed below at a dose of 100 mg following a moderate-fat breakfast:

Regimen A: Form 15 (15% Drug Load Tablet)

Regimen B: Form 18 (18% Drug Load Tablet)

Regimen C: Form 20 (20% Drug Load Tablet)

Regimen D: Form E-15 (15% Drug Load Extrudate)

Regimen E: Norvir® Soft Gelatin Capsule (Reference SGC 100 mg), the solution composition in the capsule is described in Example 9 of WO 00/74677.

The 15% Drug Load Tablet (Form 15) and 20% Drug Load (Form 20) tablets met U.S. FDA bioequivalence criteria relative to Norvir® Soft Gelatin Capsule. The 18% Drug Load (Form 18) and 15% Drug Load Extrudate (Form E-15) met bioequivalence criteria with respect to AUCₜ and AUC_{inf} relative to Norvir® Soft Gelatin Capsule at a dose of 100 mg, and the upper limits of the 90% confidence interval for Cₘₐₓ extended slightly above 1.25 for each formulation.

### EXAMPLE 2

An extrudate including 74 wt % copovidone, 10 wt % Cremophor® RH 40, 15% ritonavir and 1% colloidal anhydrous silica was analyzed by the DSC thermograph. The DSC thermogram showed no melting endotherm of crystalline ritonavir. No indication for the presence of crystalline ritonavir was observed in Raman spectra. In contrast, a characteristic peak for non-crystalline ritonavir was found in Raman spectra. Non-crystalline ritonavir can be distinguished by the characteristic peak in Raman spectra. These data suggest that the extrudate did not contain, or contained only an undetectable amount of, crystalline ritonavir.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A pharmaceutical dosage form comprising a solid dispersion or solid solution of (2S,3S,5S)-5-(N-(N-((N-methyl-N-((2-isopropyl-4-thiazolyl)methyl)amino)carbonyl)-L-valinyl)amino)-2-(N-((5-thiazolyl)methoxycarbonyl)amino)-1,6-diphenyl-3-hydroxyhexane (ritonavir) in a matrix, wherein said matrix comprises at least one pharmaceutically acceptable water-soluble polymer and at least one pharmaceutically acceptable surfactant, wherein said ritonavir is present in an amount of at least 10 wt %, based on the weight of said solid dispersion or solid solution, wherein each of said at least one pharmaceutically acceptable surfactant has an HLB value of from 12 to 18, and at least 50 percent by weight of all surfactant(s) in said solid dispersion or solid solution have an HLB value of from 12 to 18, wherein each of said at least one pharmaceutically acceptable water-soluble polymer has a T_{g} of at least 50° C., and wherein said solid dispersion or solid solution does not comprise, or comprises only an insignificant amount of, PEG.

2. The dosage form according to claim 1, wherein said at least one pharmaceutically acceptable water-soluble polymer is present in an amount of at least 50 wt %, based on the weight of said solid dispersion or solid solution.

3. The dosage form according to claim 2, wherein said at least one pharmaceutically acceptable surfactant is present in an amount of at least 5 wt %, based on the weight of said solid dispersion or solid solution.

4. The dosage form according to claim 3, wherein each of said at least one pharmaceutically acceptable water-soluble polymer is selected from the group consisting of homopolymer of N-vinyl lactam, copolymer of N-vinyl lactam, cellulose ester, cellulose ether, polyalkylene oxide, polyacrylate, polymethacrylate, polyacrylamide, polyvinyl alcohol, vinyl acetate polymer, oligosaccharide, and polysaccharide.

5. The dosage form according to claim 3, wherein each of said at least one pharmaceutically acceptable water-soluble polymer is selected from the group consisting of homopolymer of N-vinyl pyrrolidone, copolymer of N-vinyl pyrrolidone, copolymer of N-vinyl pyrrolidone and vinyl acetate, copolymer of N-vinyl pyrrolidone and vinyl propionate, polyvinylpyrrolidone, methylcellulose, ethylcellulose, hydroxyalkylcelluloses, hydroxypropylcellulose, hydroxyalkylalkylcellulose, hydroxypropylmethylcellulose, cellulose phthalate, cellulose succinate, cellulose acetate phthalate, hydroxypropylmethylcellulose phthalate, hydroxypropylmethylcellulose succinate, hydroxypropylmethylcellulose acetate succinate, polyethylene oxide, polypropylene oxide, copolymer of ethylene oxide and propylene oxide, methacrylic acid/ethyl acrylate copolymer, methacrylic acid/methyl methacrylate copolymer, butyl methacrylate/2-dimethylaminoethyl methacrylate copolymer, poly(hydroxyalkyl acrylate), poly(hydroxyalkyl methacrylate), copolymer of vinyl acetate and crotonic acid, partially hydrolyzed polyvinyl acetate, carrageenan, galactomannan, and xanthan gum.

6. The dosage form according to claim 3, wherein said ritonavir is molecularly dispersed in said matrix.

7. The dosage form according to claim 6, wherein said water-soluble polymer is copovidone, and said surfactant is polyoxyl 40 hydrogenated castor oil or macrogolglycerol hydroxystearate.

8. The dosage form according to claim 6, wherein said at least one water-soluble polymer is copovidone, and said at least one surfactant is polyoxyl 40 hydrogenated castor oil or macrogolglycerol hydroxystearate.

9. The dosage form according to claim 8, wherein said dosage form is a tablet coated with a film coating.

10. The dosage form according to claim 6, wherein said ritonavir is in an amount of at least 25 mg.

11. The dosage form according to claim 6, wherein the 90% confidence interval of the relative average Cₘₐₓ, AUC_{36 hours} or AUC_{∞} of said dosage form as compared to a reference ritonavir solution is within the range of from 80% to 125%, and wherein said reference ritonavir solution has the same absolute amount of ritonavir as said dosage form and consists of 12 wt % ethanol, 0.025 wt % butylated hydroxytoluene, 70.975 wt % oleic acid, 10 wt % ritonavir, 1 wt % water, and 6 wt % polyoxyl 35 castor oil.

12. The dosage form according to claim 3, wherein each of said at least one pharmaceutically acceptable surfactant is selected from the group consisting of polyoxyethyleneglycerol triricinoleate, polyoxyl 35 castor oil, polyoxyethyleneglycerol oxystearate, polyethylenglycol 40 hydrogenated castor oil, polyethylenglycol 60 hydrogenated castor oil, a mono fatty acid ester of polyoxyethylene (20) sorbitan, polyoxyethylene (20) sorbitan monooleate, polyoxyethylene (20) sorbitan monostearate, polyoxyethylene (20) sorbitan monopalmitate, and polyoxyethylene (20) sorbitan monolaurate.

13. The dosage form according to claim 1, wherein said ritonavir is present in an amount of from 10 to 30 wt %, said water-soluble polymer is present in an amount of from 50 to 85 wt %, and said surfactant is present in an amount of from 5 to 20 wt %, all wt % being based on the weight of said solid dispersion or solid solution.

14. The dosage form according to claim 13, wherein said water-soluble polymer is copovidone, and said surfactant is polyoxyl 40 hydrogenated castor oil or macrogolglycerol hydroxystearate.

15. The dosage form according to claim 14, wherein said solid dispersion or solid solution does not comprise, or comprises only an insignificant amount of, sorbitan monolaurate.

16. The dosage form of claim 3, wherein said dosage form further comprises another therapeutic agent.

17. The dosage form of claim 16, wherein said another therapeutic agent is an HCV protease inhibitor.

18. A process of making a pharmaceutical dosage form, said process comprising converting a mixture of ritonavir and additional ingredients into a solid dispersion or solid solution, said additional ingredients including at least one water-soluble polymer and at least one surfactant, wherein said ritonavir is present in an amount of at least 10 wt %, based on the weight of said solid dispersion or solid solution, wherein each of said at least one pharmaceutically acceptable surfactant has an HLB value of from 12 to 18, and at least 50 percent by weight of all surfactant(s) in said solid dispersion or solid solution have an HLB value of from 12 to 18, wherein each of said at least one pharmaceutically acceptable water-soluble polymer has a T_{g} of at least 50° C., and wherein said solid dispersion or solid solution does not comprise, or comprises only an insignificant amount of, PEG.

19. The process according to claim 18, further comprising grinding said solid dispersion or solid solution, and mixing the ground solid dispersion or solid solution with one or more excipients.

20. The process according to claims 19, further comprising compressing the mixture of the ground solid dispersion or solid solution and said one or more excipients into a tablet.

21. A method of treating HIV infection comprising administering to a human in need thereof a dosage form of claim 1.

22. A method for improving pharmacokinetics or increasing plasma level of a drug which is metabolized by cytochrome P450 monooxygenase, comprising administering to a human in need of such treatment a combination of said drug and a dosage form of claim 1, or a dosage form of claim 1 which further comprises said drug.

23. The method of claim 22, wherein said drug and said dosage form are administered to said human simultaneously.

24. The method of claim 22, wherein said drug and said dosage form are administered to said human sequentially.

25. A method for inhibiting cytochrome P450 monooxygenase comprising administering to a human in need thereof a dosage form of claim 1.
